(19) United States
(12) Patent Application Publication
(10) Pub. No.: US 2024/0180430 A1
(43) Pub. Date: Jun. 6, 2024

(54) HEAT DIFFUSION IMAGING SYSTEM, METHOD AND DEVICE

(71) Applicant: H.T BIOIMAGING LTD, Ganei Am (IL)

(72) Inventors: Shani TOLEDANO, Tel Aviv (IL); Hen TOLEDANO, Rishon Le-Zion (IL)

(73) Assignee: H.T BIOIMAGING LTD, Ganei Am (IL)

(21) Appl. No.: 18/284,838

(22) PCT Filed: Mar. 31, 2022
(86) PCT No.: PCT/IL2022/050345

§ 371 Date: Sep. 28, 2023

Related U.S. Application Data
(60) Provisional application No. 63/169,442, filed on Apr. 1, 2021.

(51) Int. Cl. A61B 5/01; A61B 1/00; A61B 1/015; A61B 1/04; A61B 1/12

(52) U.S. Cl. CPC A61B 5/015; A61B 1/000095; A61B 1/00126; A61B 1/015; A61B 1/046; A61B 1/128

(57) ABSTRACT

A heat diffusion imaging device is disclosed. The device comprises a thermal camera; a temperature control unit, configured to in-vivo control a temperature of a portion of an organ and an endoscope. The endoscope may include, a first array of optical fibers configured to transfer thermal IR signals in a wavelength of 7.5-14 μm, from the first end of the device to the thermal camera; and a heat delivery port, connected to the temperature control unit, located at a first end of the device. The device may further include a connector configured to connect the first array of optical fibers to the thermal camera.

### CROSS-REFERENCE TO RELATED-APPLICATIONS

This application claims the benefit of priority of U.S. Provisional Application No. 63/169,442, titled “HEAT DIFFUSION IMAGING SYSTEM, METHOD AND DEVICE”, filed Apr. 1, 2021, the contents of which are incorporated herein by reference in their entirety.

### FIELD OF THE INVENTION

The present invention relates generally to heat diffusion imaging devices and methods. More specifically, the present invention relates to heat diffusion imaging devices that include an endoscope.

### BACKGROUND OF THE INVENTION

Different cells in a living body (e.g., either a human or animal body) are distinct from one another by several properties, one of which is a thermo-physical property referred to as heat diffusivity. For example, the heat diffusivity of tumor cells is expected to be different than the heat diffusivity of healthy cells.

Accordingly, a system or a device may use this phenomenon for detecting abnormal cells in healthy tissue.

In-vivo surgical procedures, such as, laparoscopic procedures, endoscopic procedures, gastroscopic procedures, cardiac catheterization, and the like, are becoming the “process of choice” in many surgical procedures. Accordingly, a device allowing real-time detection of abnormal cells in an organ, capable of being inserted into a cavity, a duct, or a vessel during operation may have great advantages for doctors and patients alike.

### SUMMARY OF THE INVENTION

Some aspects of the invention may be directed to heat diffusion imaging device comprising: a thermal camera; a temperature control unit, configured to in-vivo control a temperature of a portion of an organ and an endoscope. In some embodiments, the endoscope may include, a first array of optical fibers configured to transfer thermal IR signals in a wavelength of 7.5-14 μm, from the first end of the device to the thermal camera; and a heat delivery port, connected to the temperature control unit, located at a first end of the device. In some embodiments, the device may further include a connector configured to connect the first array of optical fibers to the thermal camera.

In some embodiments, the thermal camera is located at a second end of the device. In some embodiments, that may include at least one black body element for bundling together the optical fibers in the array; and at least one optical lens configured to direct the thermal IR (optical) signals form the array to a thermal sensor in the thermal camera.

In some embodiments, the heating unit may include: electromagnetic (EM) radiation source providing EM radiation at a wavelength of 780-1200 nm, located at the second end of the device; and a second array of optical fibers configured transfer EM waves at the wavelength of 780-1200 nm from the EM radiation source to the first end of the device. In some embodiments, the second array of optical fibers is thermally isolated from the first array of optical fibers.

In some embodiments, the heating unit may include: a reservoir of heated fluid, located at the second end of the device; and a tube for providing the heated fluid to the first end of the device, such that, the heat delivery port is an opening in the tube.

In some embodiments, the heating unit may include: a reservoir of a thermochemical compound, such that, the heat delivery port is a delivery unit for providing the thermochemical compound to the first end of the device.

In some embodiments, the heat diffusion imaging device may further include an optical camera, located at the second end of the device, configured to receive optical signal in the visible wavelength range. In some embodiments, the heat diffusion imaging device may further include a third array of optical fibers configured to transfer signals in the visible wavelength range, from the first end of the device to the optical camera; and a connector configured to connect the third array of optical fibers to the optical camera. In some embodiments, the heat diffusion imaging device may further include an ultraviolet (UV) source for providing UV light to the first end of the device, and wherein the optical camera is further configured to take images of the portion of the object in response to the provision of UV light.

In some embodiments, the heat diffusion imaging device may further include a controller configured to: receive thermal optical data from the thermal camera; and determine locations of at least one first type of tissue and at least one second type of tissue in the portion of the organ based on the thermal optical data. In some embodiments, the controller is further configured to control the heating unit to elevate the temperature of the portion of the organ to a predetermined temperature. In some embodiments, the optical data comprises thermal IR signals received from two or more different adjacent locations in the portion of the organ, such that, the controller is further configured to extrapolate the optical data to form a continuous map of the portion of the organ.

In some embodiments, the controller is configured to: receive visible optical data in the visible wavelength range from an optical camera; and combine the visible optical data and the thermal optical data to form a single map of the portion of the organ.

In some embodiments, the controller is configured to: receive visible optical data in the visible wavelength range from an optical camera; and form a registration between the visible optical data and the thermal optical data.

In some embodiments, the organ is a moving organ, and controller is configured to: receive a stream of images, of the portion of the organ, form the thermal camera; and correct noise in the received stream of images, originated form the movement of the organ, by comparing at least two consecutive images from the stream of images.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A, 1B, 1C and 1D are illustrations of heat diffusion imaging devices according to some embodiments of the invention;

FIG. 1E is a block diagram of a heat diffusion imaging device according to some embodiments of the invention;

FIGS. 2A and 2B are illustrations of endoscopes according to some embodiments of the invention;

FIGS. 3A, 3B and 3C are illustrations of cross sections of arrays of IR optical fibers in bundles according to some embodiments of the invention;

FIGS. 4A, 4B and 4C are illustrations of cross sections of a first array of IR optical fibers and a second array of optical both include in the same bundle according to some embodiments of the invention;

FIGS. 5A, 5B and 5C are illustrations of cross sections of the first array of IR optical fibers, the second array of optical fibers and a third array of optical fibers, according to some embodiments of the invention;

FIG. 6 is an illustration of an endoscope comprising the cross section illustrated in FIG. 5C; and

FIG. 7 includes scattered temperature measurements measured using a device comprising the endoscope of FIG. 6 and a graph presenting a temperature calculated based on the scattered temperature measurements according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Aspects of the invention may be directed to an in-vivo thermal imaging device to be used prior to, during, or following an in vivo operational procedure. The operational procedure may be any laparoscopic, endoscopic, gastroscopic, and the like, that may benefit from the ability to receive, in real-time, a thermal image of an organ of interest that allows distinguishing between different tissues of the organ. Thermal imaging may allow distinguishing between two tissues having different thermal diffusivity. For example, thermal/heat diffusivity of neoplastic tissue may differ from normal tissue, due to tissue property characteristics. Accordingly, heating the examined area/organ safely up to 41.00° C. and registering the heat-decay, may enable the identification of normal, abnormal, and neoplastic (cancerous) tissues, and differentiating between them.

Therefore, in vivo device according to embodiments of the invention may include at least two components, a heating unit capable of heating the examined area/organ to the required temperature (e.g., up to 41.00° C.), and a thermal optical unit that is configured to measure the heat-decay of the examined area/organ. In some embodiments, the thermal optical unit may include at least a thermal camera and an array of optical fibers configured to transfer thermal IR signals in a wavelength of 7.5-14 μm, from the organ to the thermal camera. In some embodiments, the device may include two main portions, an in vivo portion, included in an endoscope, that is configured to be inserted into a living body cavity, duct, or vessel, and an ex-vivo portion. The endoscope may include at least a bundle that includes one or more optical fibers arrays and the ex-vivo portion may include the diagnostic sensors, power supply, controller, and the like.

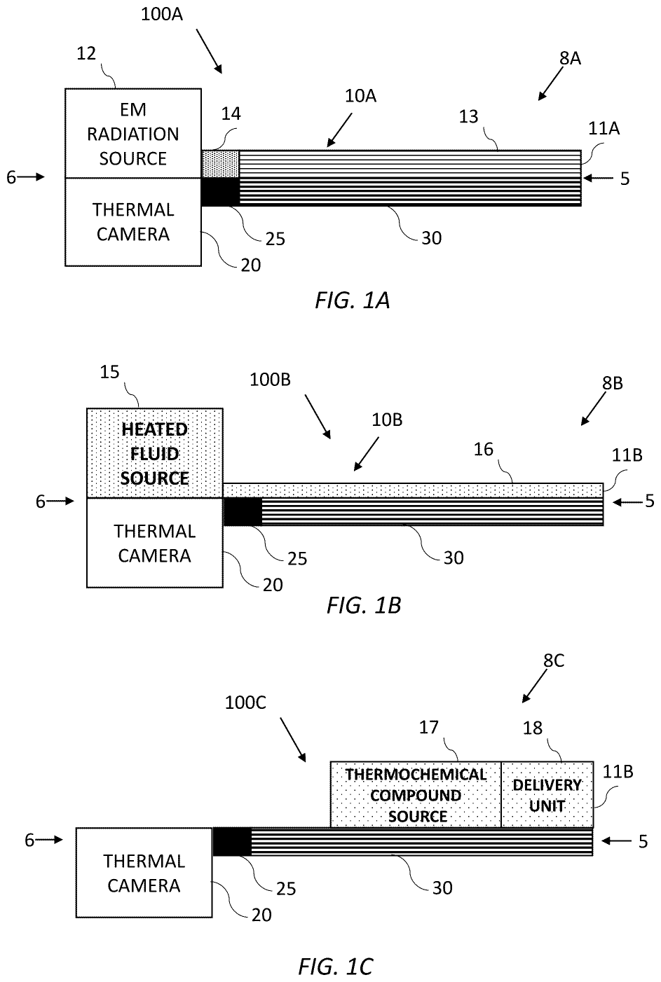

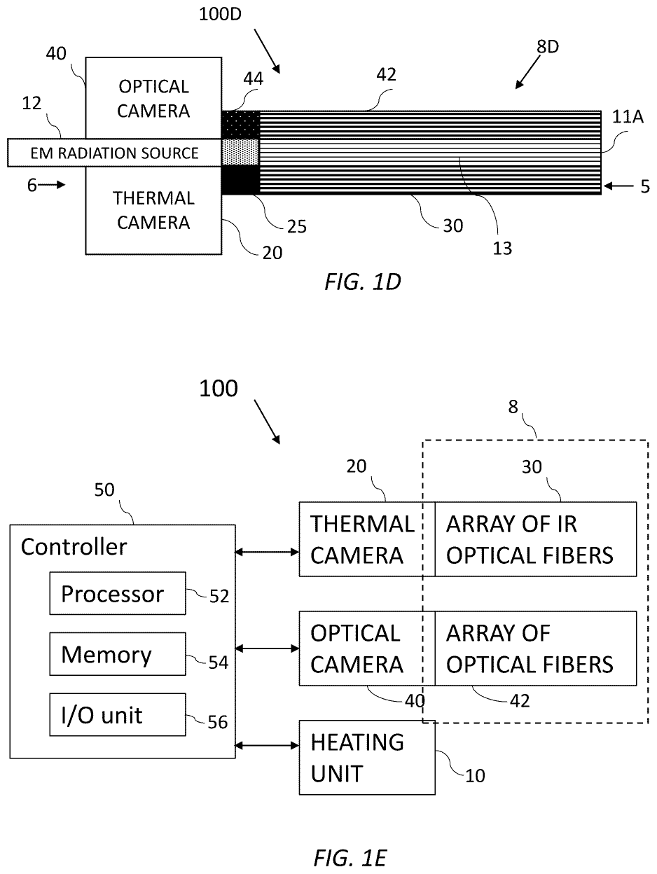

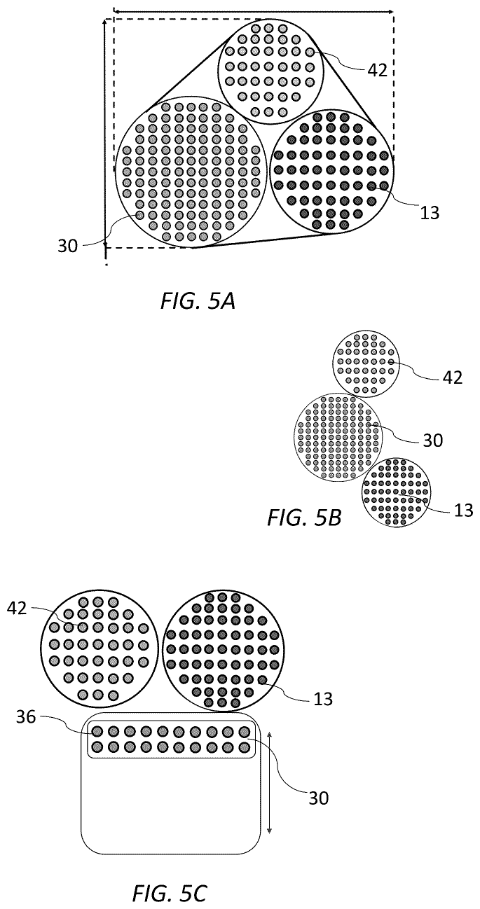

Reference is now made to FIGS. 1A, 1B, 1C, 1D, and 1E which are simplified illustrations and a block diagram of in-vivo thermal imaging devices according to some embodiments. Devices 100, 100A, 100B, 100C, and 100D may include an endoscope 8, 8A, 8B, 8C and 8D configured to be inserted into a body (e.g., human or animal) cavity, duct, or vessel. Three nonlimiting examples for endoscopes are illustrated and discussed with respect to FIGS. 2A, 2B and 6. Devices 100, 100A, 100B, 100C, and 100D may further include at least a heating unit 10, 10A, 10B, and 10C configured to in-vivo heat a portion of an organ at a first end 5 of devices 100-100D, for example, from ports 11A, 11B and 11C. Various nonlimiting examples, for specific heating units are given in FIGS. 1A-1D. Devices 100-100D may further include a thermal camera 20 located at a second end 6 of devices 100-100D and endoscope 8, 8A, 8B, 8C and 8D may include at least a first array of optical fibers 30 configured to transfer thermal infrared (IR) signals in a wavelength of 7.5-14 μm, from a first end 5 of devices 100-100D to thermal camera 20. In some embodiments, a connector 25 may connect first array 30 of IR thermal optical fibers to thermal camera 20.

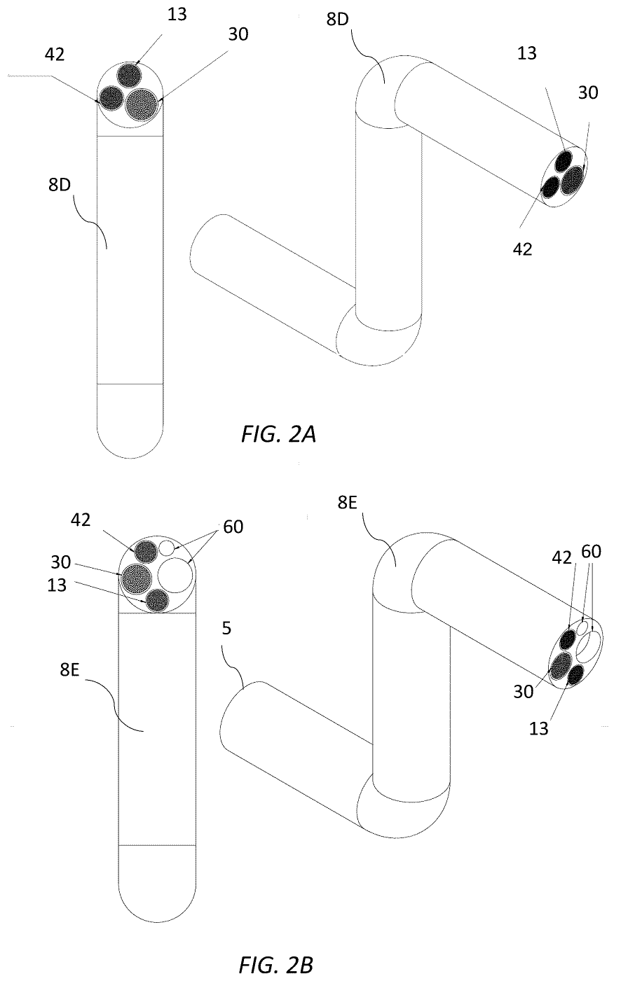

In some embodiments, thermal camera 20 may be any thermal camera known in the art. First array 30 may include a plurality of optical fibers arranged in a predetermined order within the array and bundled together, in at least one bundle. Some nonlimiting examples for such predetermined orders are given in FIGS. 3, 4, and 5. In some embodiments, the optical fibers of first array 30 may be configured to transfer thermal IR signals in the IR range, for example, at a wavelength of 7.5-14 μm.

In some embodiments, first array of fibers 30 may be connected to camera 20 via connector 25 having a shape of a small tube, made, for example, from aluminum, alloy and casting as a “black body” having emissivity of >0.98. The connector may further include one or more lenses configured to direct the IR waves transferred by first array 20 towards a sensor of camera 20. The one or more lenses may be made, for example, from selenide, zinc sulfide, and the like.

Reference is now made to FIG. 1A in which is a first nonlimiting example for heating unit 10A and an endoscope 8A of device 100A. Heating unit 10A may include an electromagnetic (EM) radiation source 12 providing EM radiation at a wavelength of 350-1200 nm, located at a second end 6 of device 100A and a second array 13 of optical fibers configured to transfer EM waves at the wavelength of 350-1200 nm from the EM radiation source 12 to first end 5 of device 100A. In some embodiments, first array 30 and second array 13 may be included in endoscope 8A. Second array 13 may include optical fibers configured transfer EM waves at the wavelength of 350-1200 nm, for example, 350-700 nm (e.g., blue light 415 nm and green light 540 nm) and 780-1200 nm. The optical fibers may be made from, silica, fluorozirconate, fluoroaluminate, chalcogenide glass and the like. A light provided by source 12 may progress in array 13 to be delivered to the portion of the organ via port 11A, which is the end of the fibers of second array 13.

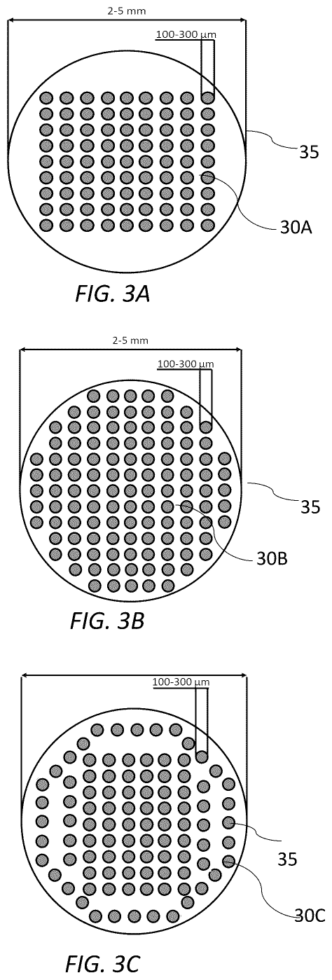

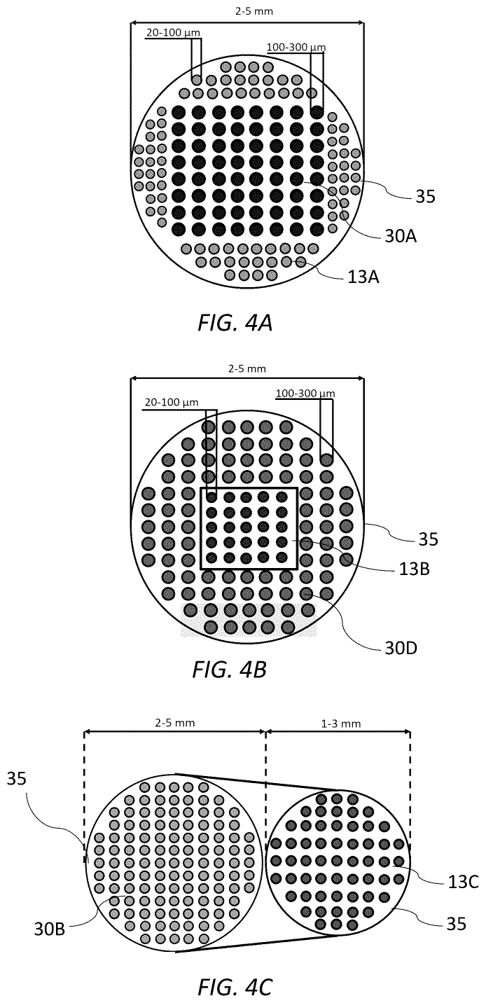

Some nonlimiting examples of the various arrangements of the fibers in second array 12 together with the fibers in first array 30 are illustrated in FIGS. 4 and 5.

Reference is made to FIG. 1B in which a second nonlimiting example for heating unit 10B and an endoscope 8B of device 100B. Heating unit 10B may include a reservoir of heated fluid 15, located at second end 6 of the device 100B. For example, reservoir of heated fluid 15 may include a heating element for heating either a gas or liquid to be delivered to a tube 16 for providing the heated fluid to first end 5 of device 100B. In some embodiments, reservoir 15 may further include a pump or a compressor for providing the fluid from reservoir 15 to pipe 16. In some embodiments, the heat delivery port is an opening 11B in tube 16. In some embodiments, first array 30 and pipe 16 may be included in endoscope 8B.

Reference is now made to FIG. 1C in which a third nonlimiting example for heating unit 10C and endoscope 8C of device 100C is illustrated. Heating unit 10C may include a reservoir/source 17 of a thermochemical compound such that a heat delivery port 11C a delivery unit 18 for providing the thermochemical compound to the first end of the device. In some embodiments, first array 30 and at least delivery unit 18 may be included in endoscope 8C. In some embodiments, reservoir/source 17 may also be included in endoscope 8C. In some embodiments, thermochemical compound source/reservoir 17 may be located near first end 5, second end 6 or any location.

In some embodiments, the in-vivo thermal imaging device may further include an optical camera. Reference is now made to FIG. 1D which is an illustration of heat diffusion imaging device 100D according to some embodiments of the invention. Device 100D may include substantially the same elements as device 100A. In some embodiments, device 100D may further include an optical camera 40, located at the second end of the device, configured to receive optical signal in the visible wavelength range and a third array 42 of optical fibers configured to transfer signals in the visible wavelength range, from the first end of the device to optical camera 40. In some embodiments, third array 42 of optical fibers may be connected to camera 40 via a connector 44. In some embodiments, connector 44 may have a shape of a small tube, made, for example, from aluminum, alloy and casting as a “black body” having emissivity of >0.98. the connector may further include one or more lenses configured to direct the visible light waves transferred by third array 42 towards a sensor of camera 40. The one or more lenses may be made, for example, from selenide, zinc sulfide and the like. In some embodiments, first array 30, second array 13 and third array 42 may all be included in endoscope 8D further illustrated in FIG. 2A Third array 42 may include a plurality of optical fibers arranged in a predetermined order within the array and bundled together, in at least one bundle. Some nonlimiting examples for such predetermined orders are given in FIGS. 2A, 2B, 5A, 5B, and FIG. 5C together with first array 30 and second array 13.

In some embodiments, heat diffusion imaging device 100 may further include an ultraviolet (UV) source (not illustrated) for providing UV light to the first end of the device, and wherein the optical camera may further be configured to take images of the portion of the object in response to the provision of UV light.

In some embodiments, devices 100-100D may further include a controller 50 illustrated in the block diagram of FIG. 1E. Controller 50 may include a processor 52 that may be any computing/calculating device, such as, a chip, a memory 54, and an input/output unit 56. Memory 54 may be any non-transitory readable medium configured to store thereon instructions and codes to be executed by processor 52. Input/output unit 56 may include any input/output device that allow processor 52 to communicate with external devices and/or users.

In some embodiments, processor 52 may be configured to control at least some of the controllable elements of devices 100-100D. For example, processor 52 may execute instructions stored on memory 54 to: receive thermal optical data from thermal camera 20; and determine locations of at least one first type of tissue and at least one second type of tissue in the portion of the organ based on the thermal optical data. In some embodiments, the optical data may include thermal optical signals received from two or more different adjacent locations in the portion of the organ, and processor 52 may further be configured to extrapolate the optical data to form a continuous map of the portion of the organ.

In another example, processor 52 may execute instructions stored on memory 54 to control heating unit 10-10C to elevate the temperature of the portion of the organ to a predetermined temperature. In yet another example, processor 52 may execute instructions stored on memory 54 to receive visible optical data in the visible wavelength range from optical camera 40 and combine the visible optical data and the thermal optical data to form a single map of the portion of the organ.

Reference is now made to FIGS. 2A and 2B which are illustrations of endoscopes according to some embodiments of the invention. FIG. 2A shows a front view and an isometric view of endoscope 8D discussed with respect to FIG. 1D.

FIG. 2B shows front view and isometric view of endoscope 8E that includes in addition to first array 30, second array 13 and third array 42 also one or more working channels 60 for delivering tools and/or materials to first end 5. For example, various operational tools, such as, obturator, punching needle, optical fiber for laser ablation, gripper and the like. Additionally or alternatively, various materials may be delivered via one or more working channels 60, for example, flushing/cleaning liquids, medications, heated/cooled fluids and the like.

Reference is now made to FIGS. 3A, 3B and 3C which are illustrations of cross sections of arrays of IR optical fibers in bundles according to some embodiments of the invention. Arrays 30A, 30B and 30C may all include a plurality of optical fibers, having diameter of 100-300 μm and any value in between, configured to transfer thermal infrared (IR) signals in a wavelength of 7.5-14 μm. Arrays 30A, 30B and 30C may differ in the order and number of optical fibers included in each array. In some embodiments, arrays 30A, 30B and 30C may each be bundled in a bundle cover 35.

In the nonlimiting example illustrated in FIG. 3A 81 fibers in a 9×9 array is bundled in bundle cover 35. In some embodiments, arrays of 6×6 to 15×15 fibers may be included in array 30A.

In the nonlimiting example illustrated in FIG. 3B, 125 optical fibers are placed in equal distances from each other inside bundle cover 35. In some embodiments, the number of optical fibers in array 30B may be between 80-250.

In the nonlimiting example illustrated in FIG. 3C 125 optical fibers may be arranged in a hexagonal formation. In some embodiments, 80-250 optical fibers may be arranged in the hexagonal formation.

Reference is now made to FIGS. 4A, 4B and 4C which are illustrations of cross sections of a first array of IR optical fibers 30A and a second array of optical fibers 13, each having diameter of 20-100 μm, for providing heat, include in the same bundle according to some embodiments of the invention. In some embodiments, both the first array and the second array may be bundled in the same bundle cover 35, as illustrated in FIGS. 4A and 4B. Alternatively, each one of the first array 30A and the second array 13A may be bundled in a separate bundle.

In the nonlimiting example illustrated in FIG. 4A first array 30A (discussed with respect to FIG. 3A) may be wrapped with second array 13A of optical fibers, each having diameter of 20-100 μm, configured transfer EM waves at a wavelength of 350-1200 nm. Array 13A may occupy the void between first array 30A and bundle cover 35.

In the nonlimiting example illustrated in FIG. 4B first array 30D may encompass second array 13B. First array 30A may include 80-250 optical fibers, having diameter of 100-300 μm, configured to transfer thermal infrared (IR) signals in a wavelength of 7.5-14 μm. Second array 13A may include 20-80 optical fibers, having diameter of 20-100 μm configured transfer EM waves at a wavelength of 350-1200 nm.

In the nonlimiting example of FIG. 4C, each one of first array 30B (discussed with respect to FIG. 3B) and second array 13C may be bundled in a separate bundle cover 35. Second array 13C may include 20-150 optical fibers, having diameter of 20-100 μm configured transfer EM waves at a wavelength of 350-1200 nm. The illustrated arrangement may allow minimizing the thermal effect of the EM energy transferred by second array 13C on the IR optical data transferred in first array 30B.

In some embodiments, a thermal isolation may be provided between the first and second arrays according to embodiments of the invention.

Reference is now made to FIGS. 5A, 5B and 5C which are illustrations of cross sections of the first array of IR optical fibers, the second array of optical fibers and a third array of optical fibers, according to some embodiments of the invention. In some embodiments, first array 30 may be any first array according to any embodiments of the invention, for example, first array 30A, 30B, 30C and 30D. In some embodiments, second array 13 may be any second array according to any embodiment of the invention, for example, second array 13A, 13B and 13C. Third array 42 may include optical fibers configured to transfer signals in the visible wavelength range.

In some embodiments, the fibers of second array 13 may be thermally isolated from the fibers of first array 30 and third array 42, to minimize the thermal effect of the EM energy transferred by second array 13. FIGS. 5A, 5B and 5C differs in the relative arrangement of the arrays. FIG. 5A shows a more compacted arrangement of the three arrays in comparison o=to FIG. 5B. In FIG. 5C first array 30 may have a form of at least one row 36 of optical fibers. In some embodiments, the at least one row 36 of optical fibers may be configured to bidirectionally move across at least a portion of the endoscope's cross section, as illustrated also in FIG. 6.

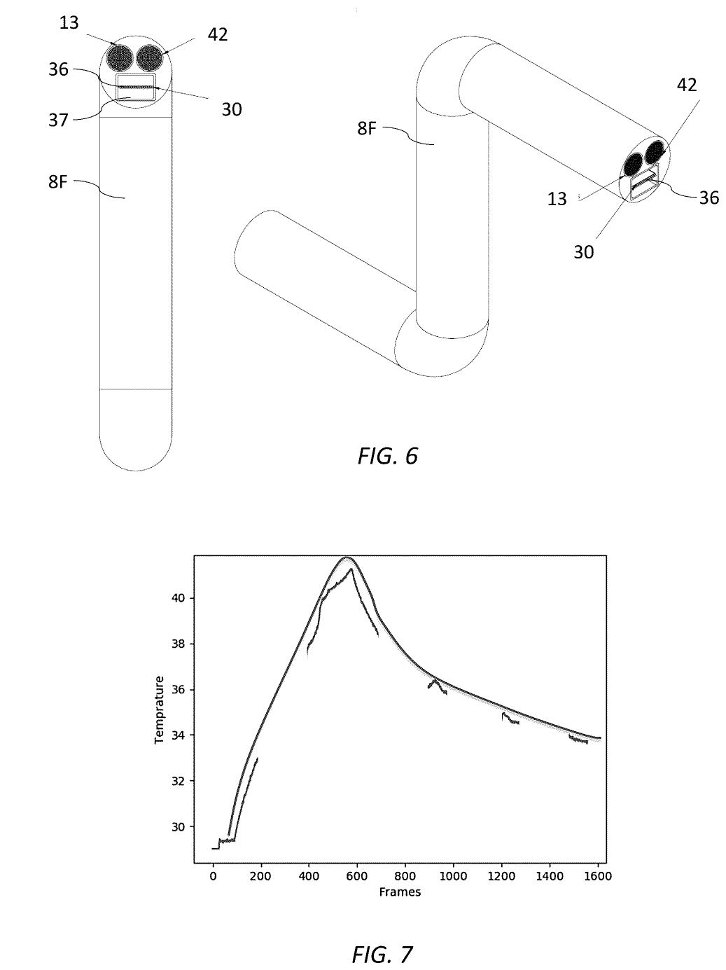

Reference is now made to FIG. 6 which is an illustration of a front view and an isometric view of an endoscope according to some embodiments of the invention. An endoscope 8E may include the arrays arrangement of FIG. 4C, when first array 30 may have a form of at least one row 36 of optical fibers configured to bidirectionally move across at least a portion of endoscope's 8E cross section. In some embodiments, endoscope 8E may include an actuator (not illustrated) configured to move at least one row 36 to cover a rectangular section 37. During the movement, at least one row 36 of optical fibers scans rectangular section 37 to detect the temporal temperature of this section. Nonlimiting example of the measured temperature received from a heat diffusion imaging device that includes endoscope 8E is given in FIG. 7.

Reference is now made to FIG. 7 which includes scattered temperature measurements measured using a device comprising endoscope 8E and a graph presenting a temperature calculated based on the scattered temperature measurements according to some embodiments of the invention. Each one of the scattered temperature measurements were taken during a single scan conducted by at least one row 36 of optical fibers. Due to the noncontinuous nature of the received data, controller, such as controller 50 may estimate/calculate the heat diffusivity in frames lacking any received data in order to create a full scan, using any known interpolation methods.

Some other aspects of the invention may be directed to a heat diffusion imaging device in which the thermal camera may be a miniature thermal camera to be included in an endoscope. In such case, the miniature camera (e.g., having a size of at most 10 mm) may be located at first end 5 of the endoscope. This arrangement may make first array 30 and connector 25 redundant. In some embodiments, the endoscope may include a communication line for delivering images captured by the miniature camera directly to the controller (e.g., controller 50).

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A heat diffusion imaging device comprising:
a thermal camera;
a temperature control unit, configured to in-vivo control a temperature of a portion of an organ;
an endoscope comprising:
a first array of optical fibers configured to transfer thermal IR signals in a wavelength of 7.5-14 μm, from the first end of the device to the thermal camera; and
a heat delivery port, connected to the temperature control unit, located at a first end of the device; and
a connector configured to connect the first array of optical fibers to the thermal camera.

2. The heat diffusion imaging device of claim 1, wherein the thermal camera is located at a second end of the device.

3. The heat diffusion imaging device of claim 1, wherein the connector comprises:
at least one black body element for bundling together the optical fibers in the array; and
at least one optical lens configured to direct the thermal IR signals form the array to a thermal sensor in the thermal camera.

4. The heat diffusion imaging device of claim 1, wherein the heating unit comprises:
electromagnetic (EM) radiation source providing EM radiation at a wavelength of 780-1200 nm, located at the second end of the device; and
a second array of optical fibers configured transfer EM waves at the wavelength of 780-1200 nm from the EM radiation source to the first end of the device.

5. The heat diffusion imaging device of claim 4, wherein the second array of optical fibers is thermally isolated from the first array of optical fibers.

6. The heat diffusion imaging device of claim 1, wherein the heating unit comprises:
a reservoir of heated fluid, located at the second end of the device; and
a tube for providing the heated fluid to the first end of the device, wherein the heat delivery port is an opening in the tube.

7. The heat diffusion imaging device of claim 1, wherein the heating unit comprises:
a reservoir of a thermochemical compound; and
wherein the heat delivery port is a delivery unit for providing the thermochemical compound to the first end of the device.

8. The heat diffusion imaging device of claim 1 further comprising:
an optical camera, located at the second end of the device, configured to receive optical signal in the visible wavelength range.

9. The heat diffusion imaging device of claim 8, further comprises:
a third array of optical fibers configured to transfer signals in the visible wavelength range, from the first end of the device to the optical camera; and
a connector configured to connect the third array of optical fibers to the optical camera.

10. The heat diffusion imaging device of claim 8, further comprising;
an ultraviolet (UV) source for providing UV light to the first end of the device,
and wherein the optical camera is further configured to take images of the portion of the object in response to the provision of UV light.

11. The heat diffusion imaging device of claim 1 further comprising:
a controller configured to:
receive thermal optical data from the thermal camera; and
determine locations of at least one first type of tissue and at least one second type of tissue in the portion of the organ based on the thermal optical data.

12. The heat diffusion imaging device of claim 11, wherein the controller is further configured to control the heating unit to elevate the temperature of the portion of the organ to a predetermined temperature.

13. The heat diffusion imaging device of claim 10,
wherein the optical data comprises thermal IR signals received from two or more different adjacent locations in the portion of the organ,
and wherein the controller is further configured to extrapolate the optical data to form a continuous map of the portion of the organ.

14. The heat diffusion imaging device of claim 10, wherein the controller is configured to:
receive visible optical data in the visible wavelength range from an optical camera; and
combine the visible optical data and the thermal optical data to form a single map of the portion of the organ.

15. The heat diffusion imaging device of claim 10, wherein the controller is configured to:
receive visible optical data in the visible wavelength range from an optical camera; and
form a registration between the visible optical data and the thermal optical data.

16. The heat diffusion imaging device of claim 10, wherein the organ is a moving organ, and the controller is configured to:
receive a stream of images, of the portion of the organ, form the thermal camera; and
correct noise in the received stream of images, originated form the movement of the organ, by comparing at least two consecutive images from the stream of images.
